(19) United States
(12) Patent Application Publication
(10) Pub. No.: US 2025/0303555 A1
(43) Pub. Date: Oct. 2, 2025

(54) DRIVE UNIT FOR A ROBOT JOINT

(71) Applicant: Eindhoven Medical Robotics B.V., Eindhoven (NL)

(72) Inventors: Anupam Vasudeva Nayak, Eindhoven (NL); Siddharth Ratnasingh Khalate, Eindhoven (NL)

(21) Appl. No.: 18/992,037

(22) PCT Filed: Jul. 3, 2023
(86) PCT No.: PCT/NL2023/050360

§ 371 Date: Jan. 7, 2025

(30) Foreign Application Priority Data
Jul. 7, 2022 (NL) 2032399

(51) Int. Cl. B25J 9/12; A61B 34/00; B25J 9/10

(52) U.S. Cl. CPC B25J 9/126; A61B 34/70; B25J 9/102

(57) ABSTRACT

Medical robots used for surgical operations comprise robot arms that have a number of joints that provide flexibility to a tool at the end of the robot arm. The joints in such a robot arm for surgical operations have drive units 1 comprising drive components 2, 3, 4, 5, 6, 7 in a frame 10. The drive unit 1 for the robot joint is complex and assembling and repairing and or replacing the unit is not easy. According to the invention the drive components 2, 3, 4, 5, 6, 7 are fixed in rings 15 where the inner shape of a ring 15 is adapted to take up and fixate a component 2, 3, 4, 5, 6, 7 and where the outer diameter of the rings 15 fits in a cylindrical hole 16 of the frame 10 and where the rings 15 are fixed to the frame 10.

Nowadays robots are used for many applications. One such application is in surgical operations. Medical robots comprise robot arms that have a number of joints that provide flexibility to a tool at the end of the robot arm. The joints in such a robot arm for surgical operations comprise drive units that need to be compact and stiff and need to be able to operate with great accuracy. Such drive units for use in a robot joint comprise drive components in a frame

Such a robot joint is known from PCT/NL2019/050217. The drive unit for the robot joint is complex and repairing and assembling the unit is not easy. It is also not easy to replace or repair a component in the drive with a different, i.e. a stronger one, a new one or to add a further component.

It is the aim of the invention to provide a drive unit for a robot joint that is easy to repair and to modify with different components.

According to the invention the components are fixed in rings where the inner shape of a ring is adapted to take up and fixate a component and where the outer diameter of the rings fits in a cylindrical hole of the frame and where the rings are fixed to the frame. The drive unit according to the invention can be adapted to different tasks easily. Different components can be fixed inside the rings, like a motor, torque-or force sensors, encoders, a brake, a gear train or a bearing unit. The inner shape of the rings is adapted to fixate the component inside the ring. The different components can be easily coupled together. No special tooling is required to assemble the drive unit. Also exchanging components of the drive unit is easy, since the rings with the components can be easily detached and re-assembled. Overall, rings attached with component (sensors, brakes, gear trains, bearings etc.) can be attached and removed without a major change in design, manufacturing and assembly procedures.

The frame, the motor components and the rings are part of a standardized modular ecosystem that can be easily re-used or modified. A motor component in a ring than represents a module that can be replaced by another module, since all modules fit within the cylindrical hole in the frame. It is not necessary to redesign a complete robot arm drive unit, but different modules can replace worn or outdated modules to restore or give added functionality. The ecosystem can also have a number of different radial and axial sizes for the frame, the accompanying rings and motor components. That way a robot arm system can be tuned to the required forces, torques and precision. Note also that no special tooling is required to assemble the drive unit. This is because each component is placed within the ring and all the rings are fixed inside the cylindrical hole in the frame.

It is difficult to prevent a drive unit to cause electrical, magnetic, or radiofrequency signal interference. To prevent such interference is especially important in a medical environment, since such interference can lead to malfunction of life saving medical devices. According to the invention the rings are made of a material that conducts electricity well. That way the rings provide extra shielding of the electric or magnetic components used in the drive unit and electromagnetic compatibility is more easily reached. The rings provide both shielding from drive unit components to other medical devices as well as shield components inside the drive unit from interference by other medical devices. Materials that conduct electricity well are metals, or materials such as graphite and conductive polymers. Minimum conductivity for the rings should be less than 10×10⁻⁸ Ωm, preferably conductivity should be around 3×10⁻⁸ Ωm.

Preferably the rings are provided with markers so that the components can be aligned with respect to each other. This makes assembling the different modules in the frame easy.

It is advantageous when different components that belong together are located in adjacent rings. For instance a magnet used for an encoder can be placed in the ring adjacent to the encoder. That way the gap between the encoder and the magnet can be determined by their axial locations in the rings and assembly is easy without difficult alignment problems inside the unit. With a marker even the exact position of the magnet with respect to the encoder can be fixed easily.

In a further embodiment the rings have at least one axial borehole and the frame has an inner flange with a diameter smaller than the hole, where the borehole is provided with a bolt that runs through the borehole and is fixed to the inner flange. That way the borehole can be used as the marker for alignments of the rings. A bolt runs through the borehole and is fixed to the flange, thus fixing the ring to the frame. The bolt can be screwed to the flange, either using a threaded hole in the flange or a nut to fasten the rings to the frame. The axial position of the inner flange in the cylindrical hole is not limited to one side of the frame. The flange can also be located for instance in the middle of the frame with components on both sides of the flange. In cases it is advantageous when the frame comprises a component not fixed via a ring. For instance the unit may be provided with a bearing that is directly fixed into the frame. That way a larger bearing can be used to provide a very precise and accurate running of the unit while transmitting larger forces or torques. It is advantageous if the bearing is provided in the inner flange.

In many cases harmonic drives are used for the gears in such drive units. Harmonic drives have a compact size (thickness/length), zero backlash properties and a reasonable reduction ratio. However, the limitations of an harmonic drive include non-linear friction and stiffness properties that limit the unit's bandwidth. Moreover those drives have a large diameter, limited reduction ratio and high cost. Preferably the component comprises a planetary gearbox, where the ring for that component comprises an internal gear for the planetary gearbox. The internal teeth can be broached on the ring's inner diameter, for instance along its width and planetary gears can be mounted around the output gear. The resulting planetary gear box is much stronger and stiffer, does not suffer from non-linear friction and is less costly than a harmonic drive and can be adapted to a wide range of reduction ratios. This way a custom planetary gear box can be made available as a module that fits inside the cylindrical hole of the frame.

In a preferred embodiment the unit comprises on one axial side of and adjacent to the motor component a motor encoder component, while on the other axial side of the motor component close to the end of an outgoing shaft a load encoder component. That way the motor steering can be done very precisely due to the close proximity of the motor encoder to the motor, while also the load on the outgoing shaft can be controlled precisely. Using different motor modules for the load-and motor encoder makes realization and assembly of such a system easy. This set up of encoders also allows to incorporate advanced control strategies including both the motor-and the load encoder.

Preferably the frame of the drive unit has a rotating outside flange connected to a motor component of the drive unit to couple it to another drive unit, where the axis of the units are perpendicular. Thus the outside flange can rotate with respect to the frame driven by the motor. It can thus rotate the next drive unit. The joints for a robot arm can thus be constructed easily by coupling a number of drive units together to make a versatile robot arm with perpendicular drive units.

Preferably the drive unit comprises at least one empty ring. That way the unit is more future proof. If there will be a need to modify or enhance the capabilities of the unit, this can be easily done by using the space made available by removing the empty ring and replacing the empty ring with a ring with a motor component with different or enhanced capabilities. The modular set-up of the robot joint with the flexible drive unit makes that easy. That way not a whole unit needs to be replaced.

Preferably a component of the drive unit comprises a local printed circuit board (PCB) corresponding to the component and the local PCB is connected via a slipring connector located at the central axis of the drive unit to a master PCB for the drive unit, where the master PCB is attached to the frame. Each component and corresponding ring, i.e. each motor module has a local PCB for reading and powering sensors, for driving a motor and providing its power, for providing other input/output data, for control and for all other electronic functions. All this information goes through slipring cables to a master PCB in a control box attached to the frame. The sliprings are located at the central axis of the drive unit. From one side of the slipring device cables run to the local PCBs. From the other side of the slipring device cables run via a central hollow space to the master PCB. The one and other side of the slipring device are able to rotate with respect to each other. That way also the electrical connections from the master PCB to the modules are compact. Preferably the master PCB comprises a computer that controls the drive unit and that is connected with a data bus and power supply. That way the drive unit is a complete system that can be controlled via the data bus, where the data bus and power supply are also connected via the slipring connector to another drive unit and a central control system for all drive units. This way the drive unit is a complete unit independent from other drive units, only communicating via the data bus with the central control system. There is the possibility to exchange a single unit for a new, repaired or updated drive unit without affecting other drive units.

The invention also deals with a frame, ring and motor module comprising a motor component in a ring according to the invention.

### DESCRIPTION FIGURES

The invention is further explained with the help of the following drawing in which

FIG. 1 shows a cross-section of a drive unit according to the invention,

FIG. 2 shows an exploded view of another drive unit according to the invention,

FIG. 3A, 3B show modules that comprise a ring and a motor component,

FIG. 4 shows a module comprising a planetary gear box,

FIG. 5 shows how the master Printed Circuit Board (PCB) is fixed to the rest of the drive unit.

The figures are for explaining only and not drawn to scale.

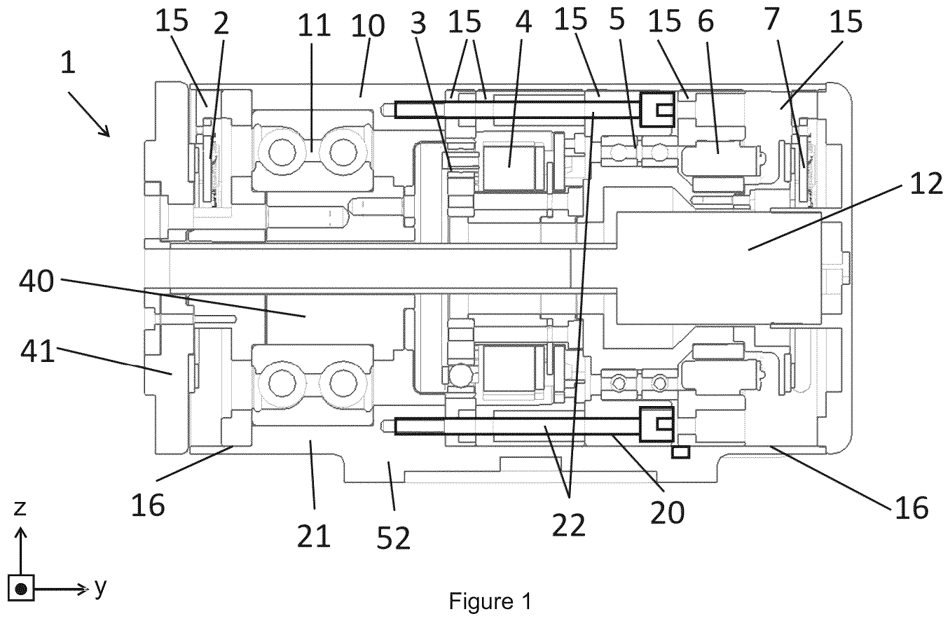

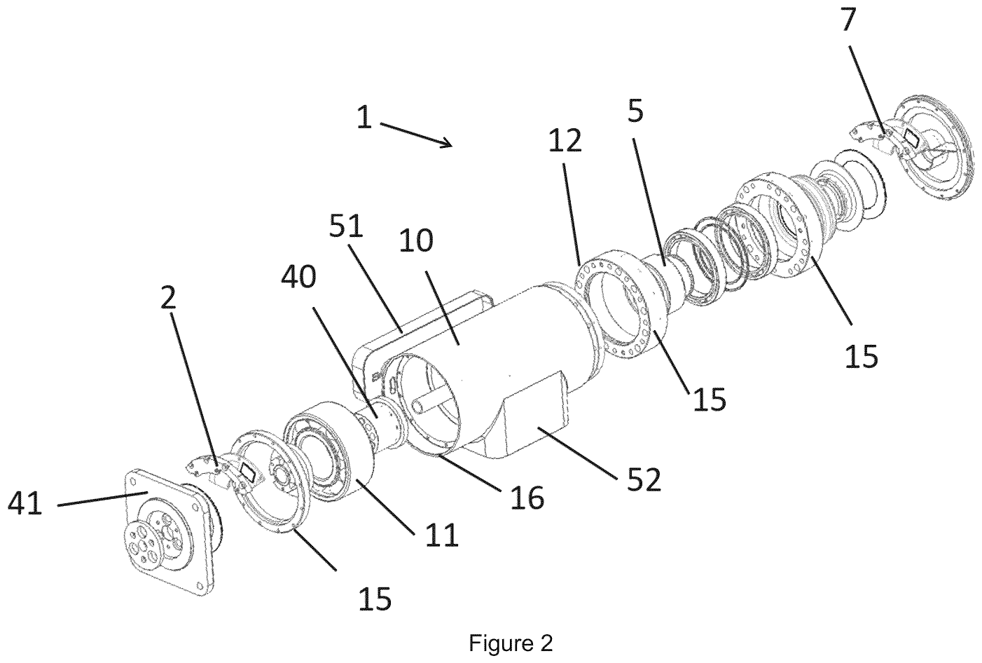

FIGS. 1 and 2 show drive units 1. Medical robots for surgical operations comprise robot arms that have a number of joints that provide flexibility to a tool at the 15 end of the robot arm. The tool is used for precision surgical operations. The joints in such a robot arm comprise drive units 1 that need to be compact and stiff and need to be able to operate with great accuracy. Such drive units 1 for use in a robot joint comprise drive components 2, 3, 4, 5, 6, 7 in a frame 10. In this case there is a load encoder 2, a harmonic drive 3, a brake 4, bearings 5, a motor 6, and a motor encoder 20 7 for the motor 6. There are further bearings 11 and a slip ring unit 12 in the drive unit 1.

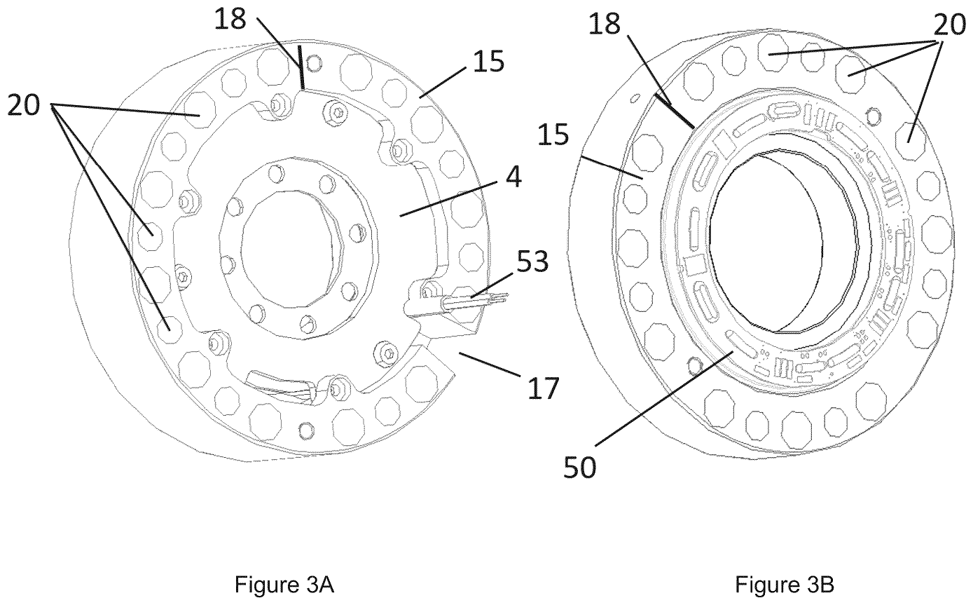

According to the invention the components 2, 3, 4, 5, 6, 7 are fixed in rings 15 where the inner shape of a ring 15 is adapted to take up and fixate a component 2, 3, 4, 5, 6, 7 and where the outer diameters of the rings 15 fit in a cylindrical hole 16 of the frame 10 and where the ring 15 can be fixed to the frame 10. The drive unit 1 according to the invention can be adapted to different tasks easily. Different components can be fixed inside the rings 15, like a motor 6, torque-or force sensors, encoders 2, 7, a brake 4, a gear train 3 (harmonic drive) or a bearing unit 11. The inner shape of the rings 15 is adapted to fixate the component 2, 3, 4, 5, 6, 7 30 inside the ring 15. The different components 2, 3, 4, 5, 6, 7 can be easily coupled together. No special tooling is required to assemble the drive unit 1. Also exchanging components of the drive unit 1 is easy, since the rings 15 with the components can be easily detached and re-assembled. The rings 15 with their components form modules with a specific functionality. Overall, modules with a specific functionality can be attached and removed without a major change in design, manufacturing and assembly procedures. Thus, it is not necessary to redesign a complete robot arm drive unit 1, but different modules can replace worn or outdated modules to restore or give added functionality. The rings 15 will have the same outer diameter, but their thickness can vary depending on the component fixed inside. Also the rings 15 do not have to be complete rings. FIG. 3A shows that a part 17 of the ring can be open. The frame 10, the motor components 2, 3, 4, 5, 6, 7 and the rings 15 are part of a standardized modular ecosystem that can be easily re-used or modified. It is possible to have different ecosystems to suit specific needs for larger or smaller drive units 1. Thus, the ecosystem can have a number of different radial and axial sizes for the frame 10, the accompanying rings 15 and motor components 2, 3, 4, 5, 6, 7. That way a robot arm system can be tuned to the required forces, torques and precision. In this case drive units 1 with an outer diameter of 73 and with 55 mm were made. The components 2, 3, 4, 5, 6, 7 are adapted to the outer diameter of the drive unit 1. In this case the larger 73 mm diameter drive units 1 have larger, stronger components, whereas the smaller drive units of 55 mm have smaller and less strong components. Thus, the dimensions of motor modules and hence the sizes of joints of a robot can be reduced or increased as applications demand. For instance the larger joints can be used near the base of the robot arm, whereas smaller units can be used near the tool at the end of the robot arm. Naturally, the frame 10 and the ring 15 dimensions will change, but the inherent assembly processes and manufacturing methods will remain the same across the modular ecosystem.

In this example the rings are made of aluminium with a minimum thickness of about 1 mm. Aluminium is a good electrical conductor with an electrical conductivity of around 2.8×10⁻⁸ Ωm at 20° C. Such rings provide excellent shielding of electrical, magnetic, or radiofrequency electromagnetic radiation giving no problems in electromagnetic compatibility tests as used for medical devices.

FIGS. 3a and 3b show how the rings 15 are provided with markers 18 so that the modules can be aligned with respect to each other and with for instance markers provided in the frame 10. This makes assembling the different modules in the frame 10 easy.

It is advantageous when different components that belong together are located in adjacent rings. For instance a magnet used for an encoder 2, 7 can be placed in its own ring adjacent to the ring 15 with the encoder 2, 7. That way the gap between the encoder 2, 7 and the magnet can be determined by their axial locations in the rings 15 and assembly is easy without difficult alignment problems inside the unit. With a marker even the exact position of the magnet with respect to the encoder 2, 7 can be fixed easily. Measurement on six actual drive units of 73 mm diameter show that for the motor encoder 7 the gap between a magnet and the corresponding encoder varies between 2.946 mm and 2.974 mm. For the load encoder 2 a similar result between 2.842 mm and 2.886 mm was obtained. These results show that the reproducibility of assembling different modules with respect to each other is excellent.

FIGS. 1 and 3 show that the rings 15 have at least one axial borehole 20 and the frame 10 has an inner flange 21 with a diameter smaller than the hole 16, where the borehole 20 is provided with a bolt 22 that runs through the borehole and is fixed to the inner flange 21. The borehole 20 and the corresponding thread in the inner flange 21 can also be used as the markers for alignments of the rings 15. The bolt 22 runs through the borehole 20 and is fixed to the inner flange 21, thus fixing the ring 15 to the frame 10. The bolt 22 can be screwed to the flange 21, either using a threaded hole in the flange or a nut to fasten the rings 15 to the frame 10. The axial position of the inner flange 21 in the cylindrical hole 16 is not limited to one side of the frame 10. The flange 21 can also be located for instance in the middle of the frame 10 with components on both sides of the flange 21. This is shown in FIG. 1 where on one side of the flange 21 there is a module with encoder 2, while on the other side of the flange 21 there are several other modules with the harmonic drive 3, brake 4, bearings 5, motor 6 and encoder 7.

In cases it is advantageous when the frame 10 comprises a component not fixed via a ring 15. For instance the unit 1 may be provided with a bearing 11 that is directly fixed into the frame 10. That way a larger bearing 11 can be used to provide a very precise and accurate running of the unit 1 while transmitting larger forces or torques. It is advantageous if the bearing 11 is provided in the inner flange 21, since there the frame is thicker than at the location of the cylindrical hole 16.

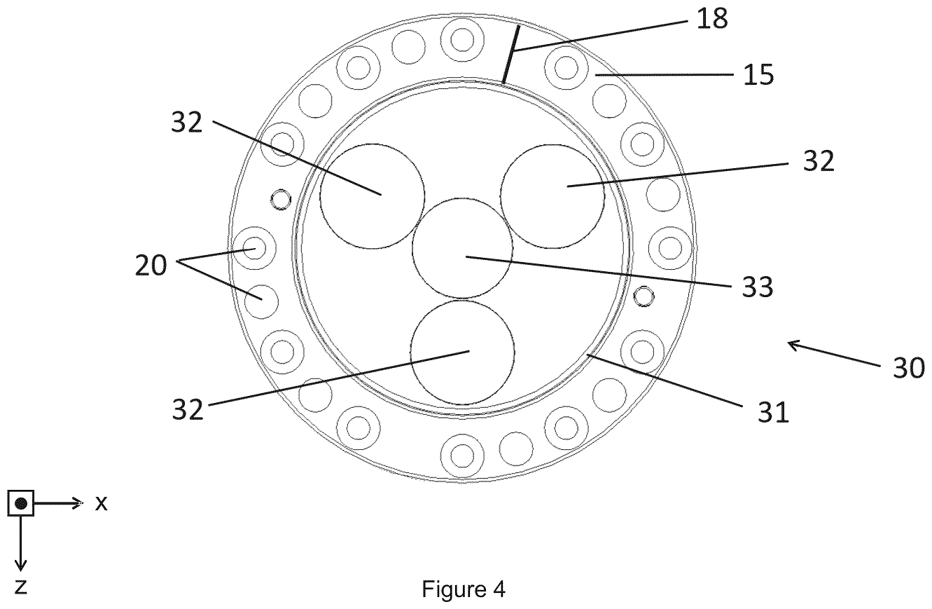

In the example of FIG. 1 a harmonic drives is used to provide a reduction in rotational speed. Harmonic drives have a compact size (thickness/length), zero backlash properties and a reasonable reduction ratio. However, the limitations of an harmonic drive include non-linear friction and stiffness properties that limit the unit's bandwidth. Moreover those drives have a large diameter, limited reduction ratio and high cost. FIG. 4 shows a module that comprises a planetary gearbox 30, also known as epicyclic gearbox. Here the ring 15 for that component comprises an internal gear 31 (ring gear) of the planetary gearbox 30. The internal teeth of the ring gear 31 can be broached on the ring's inner diameter, for instance along its width and planetary gears 32 can be mounted around the output gear (sun gear) 33. The resulting planetary gear box 30 is much stronger and stiffer, does not suffer from non-linear friction and is less costly than a harmonic drive and can be adapted to a wide range of reduction ratios. This way a custom planetary gear box 30 can be made available as a module that fits inside the cylindrical hole 16 of the frame 10.

FIG. 1 further shows that the unit 1 comprises on one axial side of and adjacent to the motor component 6 a motor encoder component 7, while on the other axial side of the motor component 6 close to the end of an outgoing shaft 40 a load encoder component 2. That way the motor steering can be done very precisely due to the close proximity of the motor encoder 7 to the motor 6, while also the load on the outgoing shaft can be controlled precisely. Using different motor modules for the load 2-and motor encoder 7 makes realization and assembly of such a system easy. This set up of encoders 2, 7 also allows to incorporate advanced control strategies including both the motor-and the load encoder.

FIGS. 1 and 2 show that the frame 10 of the drive unit 1 has a rotating output flange 41 connected to the outgoing shaft 40 of the motor component 6 to couple the drive unit 1 to another drive unit 1, where the axis of the units 1 are perpendicular. Thus the output flange 41 can rotate with respect to the frame 10 driven by the motor 6. In the robot arm the output flange 41 of a previous drive unit is connected to an outside flange 52 (see FIG. 1, 2) of a following drive unit 1. The previous drive unit 1 can thus rotate the next drive unit 1 in the chain of drive units 1 that form the robot arm. The joints for a robot arm can thus be constructed easily by coupling a number of drive units 1 together to make a versatile robot arm with perpendicular drive units 1.

Preferably the drive unit 1 comprises at least one empty ring 15 (not shown). That way the unit 1 is more future proof. If there will be a need to modify or enhance the capabilities of the unit 1, this can be easily done by using the space made available by removing the empty ring 15 and replacing the empty ring 15 with a ring 15 with a motor component with a specific capability. The modular set-up of the robot joint with the flexible drive unit 1 makes that easy. That way not a whole unit 1 needs to be replaced.

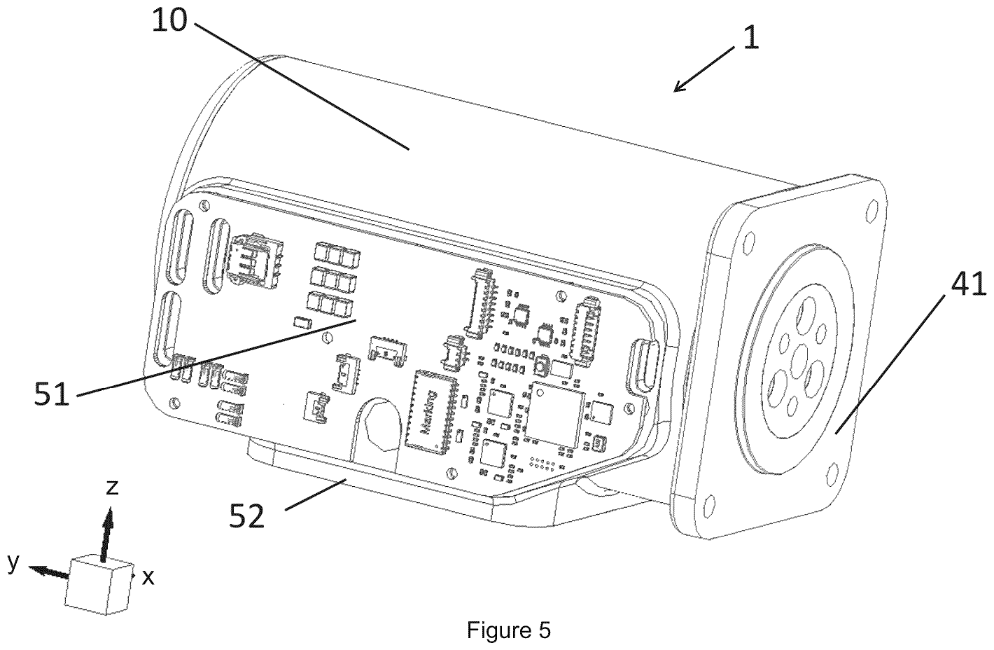

FIGS. 3b and 5 show that a component of the drive unit 1 comprises a local printed circuit board (PCB) 50 belonging to the component and the local PCB 50 is connected via a slipring connector 12 located at the central axis of the drive unit 1 to a master PCB 51 for the drive unit, where the master PCB 51 is attached to the frame 10. Each component and corresponding ring 15, i.e. each motor module has a local PCB 50 for reading and powering sensors, for driving a motor and providing its power, for providing other input/output data, for control and for all other electronic functions. All this information goes through slipring cables 53 (see FIG. 3a) to a master PCB 51 in a control box attached to the frame 10. The sliprings 12 are located at the central axis of the drive unit 1. From one side of the slipring device 12 cables 52 run to the local PCBs 50. From the other side of the slipring device 12 cables run via a central hollow space to the master PCB. The one and other side of the slipring device 12 are able to rotate with respect to each other. That way also the electrical connections from the master PCB 51 to the modules are compact. Preferably the master PCB 51 comprises a computer that controls the drive unit 1. The computer is connected with a central data bus and power supply for the robot arm. That way the drive unit 1 is a complete system that can be controlled via the data bus, where the data bus and power supply are also connected via the slipring connector 12 to another drive unit 1 and a central control system for all drive units 1. This way the drive unit 1 is a complete unit independent from other drive units 1, only communicating via the data bus with the central control system. This also gives the possibility to exchange a single unit 1 for a new, repaired or updated drive unit 1 without affecting other drive units 1.

The invention also deals with a frame, ring and motor module comprising a motor component in a ring according to the invention.

1. A drive unit for use in a robot joint with drive components in a frame characterized in that the components have different outer shapes and are fixed in rings where an inner shape of a ring is adapted to take up and fixate a component and where an outer diameter of the rings are the same and fit in a cylindrical hole of the frame and where the rings are fixed to the frame.

2. The drive unit according to claim 1 characterised in that the rings are made of a material that conducts electricity well.

3. The drive unit according to claim characterised in that the rings are provided with markers so that the components are aligned with respect to each other.

4. The drive unit according to claim 1 characterised in that different components that belong together are located in adjacent rings.

5. The drive unit according to claim 1 characterised in that the rings have at least one axial borehole and the frame has an inner flange with a diameter smaller than the hole, where the borehole is provided with a bolt that runs through the borehole and is fixed to the flange to fix the rings to the frame.

6. The drive unit according to claim 1 characterised in that the frame comprises a component not fixed via a ring.

7. The drive unit according to claim 6 characterised in that the component not fixed via a ring is a bearing, where the bearing is provided in the flange.

8. The drive unit according to claim 1 characterised in that the component comprises a planetary gearbox, where the ring for that component comprises an internal gear for the planetary gearbox.

9. The drive unit according to claim 1 characterised in that the unit comprises on one axial side of and adjacent to a motor component a motor encoder component, while on the other axial side of the motor component at the end of an outgoing shaft a load encoder component.

10. The drive unit according to claim 1 characterised in that the frame of the drive unit has a rotating outside flange connected to a motor component of the drive unit to couple it to another drive unit, where the axis of the units are perpendicular.

11. The drive unit according to claim 1 characterised in that the unit comprises at least one ring without a component.

12. The drive unit according to claim 1 characterized in that a component comprises a local printed circuit board (PCB) corresponding to the component and the local PCB is electrically connected to a master PCB for the drive unit via a slipring connector located at a central axis of the drive unit, where the master PCB is attached to the frame.

13. The drive unit according to claim 12 characterized in that the master PCB comprises a computer that controls the drive unit and that is connected with a data bus and power supply, where the data bus and power supply are also connected via the slipring connector to another drive unit and a central control system for all drive units.

14. A ring for use in a drive unit according to claim 1.

15. A frame for use in a drive unit according to claim 1.
